(19) United States
(12) Patent Application Publication
(10) Pub. No.: US 2025/0339621 A1
(43) Pub. Date: Nov. 6, 2025

(54) ROCURONIUM IN PREFILLED SYRINGES

(71) Applicant: B. Braun Melsungen AG, Melsungen (DE)

(72) Inventors: Valérie Levesque, Drummondville (CA); Juan David Delgado Maradiaga, Carignan (CA); Marie-Claude Gendron, Saint-Hyacinthe (CA)

(21) Appl. No.: 18/656,333

(22) Filed: May 6, 2024

(51) Int. Cl. A61M 5/31; A61K 31/58; A61K 47/12; A61K 47/18; A61K 47/22

(52) U.S. Cl. CPC A61M 5/3129; A61K 31/58; A61K 47/12; A61K 47/183; A61K 47/22; A61M 2207/10

(57) ABSTRACT

A method is used to manufacture a prefilled glass syringe containing a sterile pharmaceutical composition of rocuronium bromide. The prefilled syringe containing the sterile pharmaceutical composition of rocuronium bromide can be part of a kit. The sterile pharmaceutical composition of rocuronium bromide can include at least one stabilizer, a buffer system, a tonicity agent, and water. Prefilled syringes containing the sterile pharmaceutical composition of rocuronium bromide can be storage-stable for extended periods of time at room temperature.

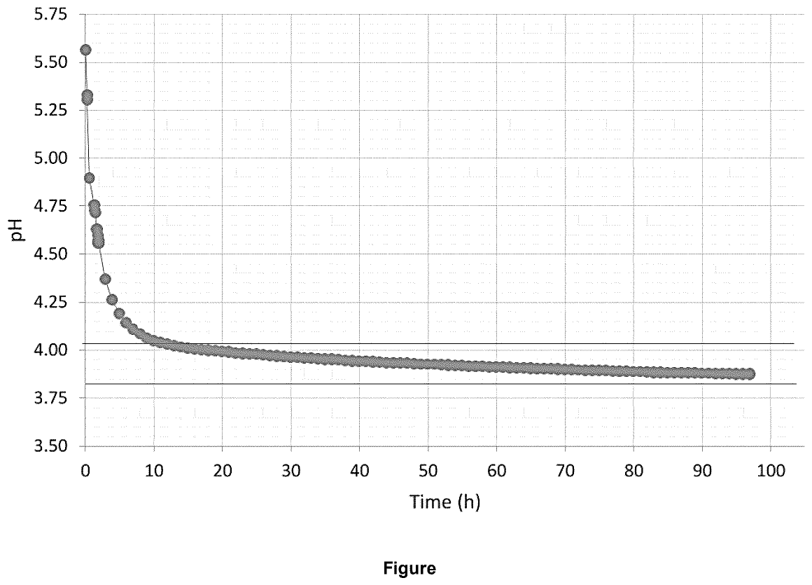

### FIELD

The present disclosure relates to a method for manufacturing a prefilled glass syringe comprising a pharmaceutical composition of rocuronium bromide, wherein said prefilled syringe can be stored at room temperature, a prefilled glass syringe prepared by said method, and a kit comprising said prefilled syringe, a plunger rod, and a carton box as secondary packaging.

### BACKGROUND

Anesthesia is typically defined as the elimination of certain body functions of a patient so that diagnostic or surgical procedures can be tolerated. Traditionally, anesthesia comprises the components of pain relief (analgesia), loss of consciousness (hypnosis), loss of vegetative functions and muscle relaxation (paralysis). These effects can be obtained from a single drug which alone provides the desired combination of effects, or a combination of drugs (such as hypnotics, sedatives, paralytics and analgesics) to achieve very specific combinations of effects.

Most currently available neuromuscular blocking agents comprise a quaternary ammonium structure, i.e. have a cationic scaffold. Such a structure allows for binding to the postsynaptic nicotinic acetylcholine receptor, thereby inhibiting or interfering with the binding of acetylcholine to the receptor finally leading to muscle relaxation.

One example of such compound is rocuronium which is an amino-steroid non-depolarizing neuromuscular blocker used in modern anesthesia to facilitate tracheal intubation through skeletal muscle relaxation.

Typically, such neuromuscular blocking agents are applied by intravenous injection. Usually, this requires dissolving said neuromuscular blocking agent that is provided, e.g. in the form of a freeze-dried powder containing the active ingredient and excipients, in a solvent containing water for injection and optionally co-solvents. However, such procedure does not only impose medical professionals with a high effort of preparing an injectable formulation and the device for injecting the formulation, but also bears the risk of medication errors as a consequence of wrong dilution resulting in a too high or too low dosage. Ensuring the correct dosage of the drug is crucial to avoid under-dosing or overdosing the patients. Even a small error in dosage can have significant consequences for patient safety and/or treatment effectiveness.

Additionally, maintaining sterility throughout the preparation of an injectable formulation and filling said injectable formulation into a device for injection, such as a syringe, is vital to prevent contamination and reduce the patient's risk of infections. Any breach in aseptic technique during handling, mixing, or transferring of the formulation would compromise patient safety.

Addressing these difficulties in a clinical setup requires strict adherence to protocols, ongoing training, quality assurance measures, and a commitment to patient safety throughout the preparation process.

On the other hand, manufacturing prefilled syringes comprising ready-to-inject formulations of rocuronium bromide proved to be difficult, if not impossible.

This is because rocuronium is known to be chemically instable. Specifically, pharmaceutically acceptable salts of rocuronium are prone to hydrolysis of the acetate ester contained in the molecule. This is particularly true when the pharmaceutically acceptable salt of rocuronium is stored in water, especially in the presence of acids or bases which are commonly known to catalyze or promote ester hydrolysis reactions.

Therefore, prefilled syringes containing an injectable formulation of rocuronium have a short shelf-life and hence must be used shortly after preparation or must be discarded. This is highly inconvenient and costly. In particular, it is thought that prefilled syringes do not provide for sufficient chemical stability of rocuronium bromide.

Therefore, there is still a need for prefilled syringes comprising a sterile pharmaceutical composition of rocuronium bromide and methods for producing the same, whereas the prefilled syringes have a prolonged shelf-life, even when stored at room temperature.

### SUMMARY

Therefore, it is an object of the present disclosure provide a method for manufacturing ready-to use prefilled syringes comprising a sterile pharmaceutical composition of rocuronium bromide, that is both cost efficient and produces prefilled syringes that can be stored at room temperature over a long time period, i.e. that have a long shelf-life, and do not require refrigeration.

The inventors of the present disclosure have surprisingly found that prefilled glass syringes comprising a sterile pharmaceutical composition of rocuronium bromide having a prolonged shelf-life, even at room temperature or elevated temperature, can be prepared by the method according to the present disclosure. Surprisingly, it has been found that a buffered solution of rocuronium bromide undergoes an automatic adjustment of pH upon stirring the formulation without addition of acid or base. Such formulation can be placed in a glass syringe. Surprisingly, the buffered solution of rocuronium bromide remains chemically stable, i.e. the amount of rocuronium bromide does not or substantially not decrease over time, even though the rocuronium bromide is dissolved in water and stored in a glass syringe.

According to a first aspect, the present disclosure relates to A method for manufacturing a prefilled glass syringe comprising a sterile pharmaceutical composition of rocuronium bromide, the method comprising the following steps:
S1: Providing a first amount of water for injection;
S2: Dissolving at least one stabilizer in the water for injection provided in step S1 to provide a stabilizer solution;
S3: Dissolving a buffer system in the stabilizer solution obtained from step S2 to provide a buffered stabilizer solution;
S4: Dissolving rocuronium bromide in the buffered stabilizer solution obtained from step S3 and adding water for injection in an amount to provide a buffered rocuronium bromide solution, wherein the buffered rocuronium composition comprises rocuronium in an amount of about 10 mg/mL;
S5: Sterilizing the buffered rocuronium composition obtained from step S5 to provide a sterile pharmaceutical composition of rocuronium bromide;
S6: Filling the sterile pharmaceutical composition of rocuronium bromide obtained from step S5 into a glass syringe under aseptic conditions to provide a glass syringe comprising the sterile pharmaceutical composition of rocuronium bromide;
S7: Blanketing the glass syringe comprising the sterile pharmaceutical composition of rocuronium bromide obtained from step S6 with an inert gas and inserting a plunger into said glass syringe to provide the prefilled glass syringe comprising the sterile pharmaceutical composition of rocuronium bromide.

According to a second aspect, the present disclosure relates to a prefilled glass syringe comprising a sterile pharmaceutical composition of rocuronium bromide obtained by the method according to the first aspect of the present disclosure.

According to a third aspect, the present disclosure relates to a kit comprising
i) the prefilled glass syringe comprising a sterile pharmaceutical composition of rocuronium bromide according the second aspect of the present disclosure or prepared by the method according to the first aspect of the present disclosure,
ii) a plunger rod, and
iii) a carton box as secondary packaging.

### BRIEF DESCRIPTION OF THE DRAWING

The FIGURE shows the development of the Ph value over time during stirring the mixing the buffered rocuronium composition in example 2.

### DETAILED DESCRIPTION

This detailed description is intended only to acquaint others skilled in the art with the present disclosure, its principles, and its practical application so that others skilled in the art may adapt and apply the present disclosure in its numerous forms, as they may be best suited to the requirements of a particular use. This description and its specific examples are intended for purposes of illustration only. This present disclosure, therefore, is not limited to the embodiments described in this patent application, and may be variously modified.

The present disclosure is described in the following in more detail, exemplified by preferred embodiments and embodiment examples. However, it is understood that the scope of the present disclosure is not limited to the preferred embodiments and embodiment examples.

The present disclosure, in very general terms, relates to three aspects, namely a first aspect being directed to a method for manufacturing a prefilled glass syringe comprising a sterile pharmaceutical composition of rocuronium bromide, a second aspect being directed to a prefilled glass syringe comprising a sterile pharmaceutical composition of rocuronium bromide obtained by the method according to the first aspect of the present disclosure, and a third aspect being directed to a kit comprising said prefilled syringe according to the second aspect of the present disclosure or prepared by said method according to the first aspect of the present disclosure.

### The Method for Manufacturing a Prefilled Syringe

According to a first aspect, the present disclosure relates to a method for manufacturing a prefilled glass syringe comprising a sterile pharmaceutical composition of rocuronium bromide, the method comprising the following steps:
S1: Providing a first amount of water for injection;
S2: Dissolving at least one stabilizer in the water for injection provided in step S1 to provide a stabilizer solution;
S3: Dissolving a buffer system in the stabilizer solution obtained from step S2 to provide a buffered stabilizer solution;
S4: Dissolving rocuronium bromide in the buffered stabilizer solution obtained from step S3 and adding water for injection in an amount to provide a buffered rocuronium bromide solution, wherein the buffered rocuronium composition comprises rocuronium in an amount of about 10 mg/mL;
S5: Sterilizing the buffered rocuronium composition obtained from step S5 to provide a sterile pharmaceutical composition of rocuronium bromide;
S6: Filling the sterile pharmaceutical composition of rocuronium bromide obtained from step S5 into a glass syringe under aseptic conditions to provide a glass syringe comprising the sterile pharmaceutical composition of rocuronium bromide;
S7: Blanketing the glass syringe comprising the sterile pharmaceutical composition of rocuronium bromide obtained from step S6 with an inert gas and inserting a plunger into said glass syringe to provide the prefilled glass syringe comprising the sterile pharmaceutical composition of rocuronium bromide.

Each step of the method of the first aspect of the present disclosure is described in the following in more detail, exemplified by preferred embodiments and embodiment examples.

### Step S1: Providing a First Amount of Water for Injection

In a first step of the method according to the present disclosure, a first amount of water for injection is provided.

Said first amount of water may be provided in any container that is suitable for carrying out the further steps of preparing a sterile pharmaceutical composition of rocuronium bromide, that are steps S2 to S5. Said container is also referred to as the manufacturing container. All subsequent steps S2 to S5 may be performed in said manufacturing container.

According to a preferred embodiment of the present disclosure, the first amount of water for injection is provided in a formulation bag, a reactor, such as a stirred tank reactor, or the like. In other words, the manufacturing container may be a formulation bag, a reactor, such as a tank reactor, or the like.

The container may comprise or consist of any material that is compatible with the sterile pharmaceutical composition of rocuronium bromide prepared in step S4 and that withstands the sterilization process (step S5). Hence, it is preferable that the manufacturing container comprises or is made of a plastic with high chemical resistance and high melting point, a metal, and/or glass. If the container comprises several layers of different materials, it is essential to ensure that the layer that is in contact with the pharmaceutical composition is chemically compatible with said pharmaceutical composition and its components, e.g. does not react with the pharmaceutical composition or its components.

According to a preferred embodiment of the present disclosure, the container comprises a plastic. According to another preferred embodiment of the present disclosure, the manufacturing container comprises or consists of ethylene-vinyl acetate (EVA) or polyethylene (PE), such as ultra low density polyethylene (ULDPE) or high density polyethylene (HDPE), or silicone, such as platinum cured silicone. A non-limiting example for such manufacturing container is the formulation bag marked under the registered trademark FLEXEL® for single-use bioprocessing bags (5 L-50 L; obtained from Sartorius Stedim Biotech GmbH, Goettingen, Germany; or Sartorious Stedim North America, Inc., Bohemia, NY, USA). A non-limiting example for a material that is silicone, such as platinum cured silicone, is the material marketed under the registered trademark TUFLUX® SIL (obtained from Sartorius Stedim Biotech GmbH, Goettingen, Germany, or Sartorious Stedim North America, Inc., Bohemia, NY, USA).

It has been found that all above-mentioned materials are compatible with the pharmaceutical composition as disclosed herein.

Therefore, it is advantageous that the manufacturing container consists of any one of said materials, or comprises any one of said materials, wherein the pharmaceutical composition is in direct contact with a layer of said material. In other words, the manufacturing container at least comprises a layer of material that is compatible with the pharmaceutical composition as disclosed herein on the innermost surface of the manufacturing container, said layer being in direct contact with the pharmaceutical composition as disclosed herein.

According to a preferred embodiment, the manufacturing container is further equipped with a magnetic stirrer, such as a magnetic stirring rod. Said magnetic stirrer aids the further steps of dissolving the components of the pharmaceutical composition.

According to a preferred embodiment of the first aspect of the present disclosure, the first amount of water for injection amounts for about 20% (V/V) to about 80% (V/V) of the volume of the sterile pharmaceutical composition of rocuronium bromide obtained in step S5. According to another preferred embodiment of the first aspect of the present disclosure, the first amount of water for injection amounts for about 30% (V/V) to about 70% (V/V) of the volume of the sterile pharmaceutical composition of rocuronium bromide obtained in step S5. According to another preferred embodiment of the first aspect of the present disclosure, the first amount of water for injection amounts for about 40% (V/V) to about 60% (V/V) of the volume of the sterile pharmaceutical composition of rocuronium bromide obtained in step S5. According to another preferred embodiment of the first aspect of the present disclosure, the first amount of water for injection amounts for about 45% (V/V) to about 55% (V/V) of the volume of the sterile pharmaceutical composition of rocuronium bromide obtained in step S5. According to a further preferred embodiment of the first aspect of the present disclosure, the first amount of water for injection amounts for about 50% (V/V) of the volume of the sterile pharmaceutical composition of rocuronium bromide obtained in step S5.

It is understood that such first amount of water is both suitable for dissolving the components of the pharmaceutical composition, that are rocuronium bromide, a buffer, and a stabilizer. At the same time, smaller volumes of fluids can be handled more easily. Furthermore, the exact volume of the final pharmaceutical composition and concentrations of the components can be adjusted more accurately.

According to another preferred embodiment of the present disclosure, step S1 further comprises sparging the water for injection with an inert gas.

According to another preferred embodiment of the present disclosure, step S1 further comprises sparging the water for injection with nitrogen for at least 15 min. According to another preferred embodiment of the present disclosure, sparging is performed at a pressure of about 5 psi (about 345 mbar).

According to another preferred embodiment of the present disclosure, the water for injection provided in step S1 has an oxygen content of about 2 ppm or less.

Thereby, it is assured that rocuronium bromide or any other component added into said first amount of water for injection cannot react with oxygen, and thus decompose, during the manufacturing of the sterile pharmaceutical composition of rocuronium bromide as shown in example 1.

Furthermore, oxygen exposure can cause changes in the composition of the formulation, leading to alterations in pH, color, or other physical properties. These changes may affect the overall quality and appearance of the injectable formulation. By controlling oxygen levels, the integrity of the sterile pharmaceutical composition of rocuronium bromide can be maintained and consistency in product characteristics is ensured.

If the oxygen content exceeds 2 ppm, it might also promote microbial growth in pharmaceutical formulations. Low oxygen environments inhibit the growth of aerobic microorganisms, reducing the risk of contamination during manufacture and storage. This is critical for maintaining the sterility and safety of injectable medications.

### Step S2: Dissolving at Least One Stabilizer in the Water for Injection Provided in Step S1 to Provide a Stabilizer Solution

In a second step (S2) of the method according to the present disclosure, at least one stabilizer is dissolved in the water for injection provided in step S1 to provide a stabilizer solution.

The inventors of the present disclosure have surprisingly found that it is highly advantageous for the stability of rocuronium bromide in the pharmaceutical composition to dissolve the at least one stabilizer before adding the further components of the pharmaceutical composition, that are buffer and rocuronium bromide. As con be concluded from Table 3 in Example 1, the pharmaceutical composition only exhibits a minor amount of impurities after preparation, if the at least one stabilizer is dissolved as a second step of the manufacturing method. Therefore, it is understood that rocuronium bromide is efficiently protected against degradation by dissolving the at least one stabilizer in the water for injection provided in step S1 to provide a stabilizer solution as a second step (S2) of the manufacturing method according to the first aspect of the present disclosure.

The at least one stabilizer may be any chemical compound or mixture of compounds that efficiently stabilizes rocuronium bromide against chemical degradation in an aqueous solution. In other words, the at least one stabilizer increases the chemical stability of rocuronium bromide in aqueous solution and hence increases the shelf-life.

According to a preferred embodiment of the present disclosure, the at least one stabilizer is four stabilizers or less. According to another preferred embodiment of the present disclosure, the at least one stabilizer is three stabilizers. According to another preferred embodiment of the present disclosure, the at least one stabilizer is two stabilizers. In other words, it may be provided that the at least one stabilizer is a mixture of two, three, or four compounds, each of which efficiently stabilizes rocuronium bromide against chemical degradation in an aqueous solution.

According to a preferred embodiment of the present disclosure, the at least one stabilizer is a single compound or stabilizer.

According to a preferred embodiment of the present disclosure, the at least one stabilizer is gluconolactone.

The inventors of the present disclosure have surprisingly found that the gluconolactone is particularly efficient in stabilizing rocuronium bromide from chemical degradation or decomposition during manufacturing (cf. example 1), sterilization (cf. example 3), and storage (cf. example 4).

According to a preferred embodiment of the present disclosure, the at least one stabilizer is added in an amount so that the buffered rocuronium composition obtained from step S4 comprises the at least one stabilizer in an amount of at least about 10 mg/mL. According to another preferred embodiment of the present disclosure, the at least one stabilizer is added in an amount so that the buffered rocuronium composition obtained from step S4 comprises the at least one stabilizer in an amount of at least about 15 mg/mL. According to another preferred embodiment of the present disclosure, the at least one stabilizer is added in an amount so that the buffered rocuronium composition obtained from step S4 comprises the at least one stabilizer in an amount of at least about 20 mg/mL.

According to a further preferred embodiment, the at least one stabilizer is added in an amount so that the buffered rocuronium composition obtained from step S4 comprises the at least one stabilizer in an amount of about 25 mg/mL.

The inventors of the present disclosure have surprisingly found that it is highly advantageous for the stability of rocuronium bromide in the pharmaceutical composition to dissolve the at least one stabilizer in the above-mentioned concentration. As can be concluded from Table 3 in Example 1, the pharmaceutical composition only exhibits a minor amount of impurities after preparation, if the at least one stabilizer is dissolved as a second step of the manufacturing method in the specified amounts. Therefore, it is understood that rocuronium bromide is efficiently protected against degradation by dissolving the at least one stabilizer in the water for injection provided in step S1 to provide a stabilizer solution as a second step (S2) of the manufacturing method according to the first aspect of the present disclosure.

The same effect is observed during sterilization (cf. example 3), and during storage (cf. example 4): A sufficient chemical stability of rocuronium bromide is observed for the compositions contained in a glass syringe prepared according to the method of the first aspect of the present disclosure.

The at least one stabilizer may also be a polyol. Said polyol is characterized in that the polyol has at least two hydroxyl (OH) groups, but does not comprise a COOX group, wherein X is hydrogen or a pharmaceutically acceptable cation. In other words, the polyol is an organic polyol with at least two hydroxyl groups, but without an organic carboxylic acid or acid salt group.

According to a preferred embodiment of the present disclosure, the polyol is a sugar alcohol, or a monosaccharide, or a disaccharide, or a polysaccharide.

According to another preferred embodiment of the present disclosure, the polyol is a sugar alcohol of formula I

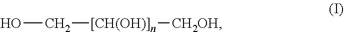

wherein n is an integer of from 1 to 10. According to another preferred embodiment of the present disclosure, polyol is a sugar alcohol of the formula I, wherein n is an integer of from 2 to 6. According to another preferred embodiment of the present disclosure, the polyol is a sugar alcohol of formula I, wherein n is an integer of from 3 to 5. According to a further preferred embodiment of the present disclosure, the polyol is a sugar alcohol of formula I wherein n is 4.

According to another further preferred embodiment of the present disclosure, the polyol is a sugar alcohol selected from the group consisting of glucose, glycerol, sorbitol, erythritol, threitol, arabitol, xylitol, ribitol, galactitol, fucitol, iditol, inositol, volemitol, isomalt, maltitol, and lactitol. According to a further preferred embodiment of the present disclosure, the at least one stabilizer is mannitol, glycerol, or sorbitol.

According to another preferred embodiment of the present disclosure, the polyol is a monosaccharide of the formula II

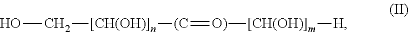

or an intramolecular lactole thereof, wherein n is an integer of from 1 to 10, and wherein m is an integer of from 0 to 2. According to another preferred embodiment of the present disclosure, the polyol is a monosaccharide of the formula II, wherein n is an integer in the range of from 2 to 6, and wherein m is an integer on the range of from 0 to 2. According to another preferred embodiment of the present disclosure, the polyol is a monosaccharide of the formula II, wherein n is 3 or 4, and wherein m is an integer on the range of from 0 to 2.

According to a further preferred embodiment of the present disclosure, the polyol is a monosaccharide of the formula II, wherein n is an integer in the range of from 2 to 6, and wherein m is 0 or 1. According to a further preferred embodiment of the present disclosure, the polyol is a monosaccharide of the formula II, wherein n is 3 or 4, and wherein m is 0 or 1.

According to another preferred embodiment of the present disclosure, the polyol is a monosaccharide of formula II, wherein the number of carbon atoms x is an integer in the range of from 3 to 14. According to another preferred embodiment of the present disclosure, the polyol is a monosaccharide of formula II, wherein the number of carbon atoms x is an integer in the range of from 3 to 10. According to another preferred embodiment of the present disclosure, the polyol is a monosaccharide of formula II, wherein the number of carbon atoms x is an integer in the range of from 3 to 8. According to another preferred embodiment of the present disclosure, the polyol is a monosaccharide of formula II, wherein the number of carbon atoms x is an integer in the range of from 3 to 7.

According to another preferred embodiment of the present disclosure, the polyol is a monosaccharide of formula II, wherein the number of carbon atoms x is 3. In other words, the polyol is a monosaccharide of formula II, wherein n is 1 and m is 0.

According to another preferred embodiment of the present disclosure, the polyol is a monosaccharide of formula II, wherein the number of carbon atoms x is 4. In other words, the polyol is a monosaccharide of formula II, wherein n is 2 and m is 0, or wherein n is 1 and m is 1.

According to another preferred embodiment of the present disclosure, the polyol is a monosaccharide of formula II, wherein the number of carbon atoms x is 7. In other words, the polyol is a monosaccharide of formula II, wherein n is 5 and m is 0, or wherein n is 4 and m is 1, or wherein n is 3 and m is 2.

According to another preferred embodiment of the present disclosure, the polyol is a monosaccharide of formula II, wherein the number of carbon atoms x is 8. In other words, the polyol is a monosaccharide of formula II, wherein n is 6 and m is 0, or wherein n is 5 and m is 1, or wherein n is 4 and m is 2.

According to a further preferred embodiment of the present disclosure, the polyol is a monosaccharide of formula II, wherein the number of carbon atoms x is 5. In other words, the polyol is a monosaccharide of formula II, wherein n is 3 and m is 0, or wherein n is 2 and m is 1, or wherein n is 1 and m is 2. According to another further preferred embodiment of the present disclosure, the polyol is selected from the group consisting of arabinose, lyxose, ribose, xylose, arabinose, ribulose, and xylolose.

According to a further preferred embodiment of the present disclosure, the polyol is monosaccharide of the formula II, wherein the number of carbon atoms x is 6. In other words, the polyol is a monosaccharide of formula II, wherein n is 4 and m is 0, or wherein n is 3 and m is 1, or wherein n is 2 and m is 2. According to a further preferred embodiment of the present disclosure, the polyol is selected from the group consisting of allose, altrose, glucose, mannose, gulose, idose, galactose, talose, psicose, fructose, sorbose, tagatose, and glutose.

According to a preferred embodiment of the present disclosure, the polyol is selected from the group consisting of glyceraldehyde, erythrose, threose, erythrulose, arabinose, lyxose, ribose, xylose, arabinose, ribulose, xylolose, allose, altrose, glucose, mannose, gulose, idose, galactose, talose, psicose, fructose, sorbose, tagatose, glutose, sedoheptulose, mannoheptulose, and glycerol-manno-heptose.

According to a further preferred embodiment of the present disclosure, the polyol is selected from the group consisting of glucose, fructose, galactose, and mixtures thereof.

According to a further preferred embodiment of the present disclosure, the at least one stabilizer is selected from the group consisting of glucose, fructose, galactose, and mixtures thereof. According to a further preferred embodiment of the present disclosure, the at least one stabilizer is glucose. According to a further preferred embodiment of the present disclosure, the at least one stabilizer is fructose. According to a further preferred embodiment of the present disclosure, the at least one stabilizer is galactose.

Generally, it is understood that the at least one stabilizer is provided in an amount that efficiently protects rocuronium bromide from chemical decomposition during storage.

As such, the at least one stabilizer is added in an amount that ensures that the sterile pharmaceutical composition of rocuronium bromide comprised in the prefilled syringe contains at least 9.8 mg/mL of rocuronium bromide after storage at 25° C. for up to 6 months, as determined by HPLC.

Additionally, the at least one stabilizer is added in an amount that ensures that the sterile pharmaceutical composition of rocuronium bromide comprised in the prefilled syringe contains at most about 1.3% of total impurities after storage at 25° C. for up to 6 months, as determined by HPLC.

The inventors of the present disclosure have surprisingly found that the prefilled syringes prepared by the method according to the first aspect of the present disclosure have a prolonged shelf-life of at least 6 months. Example 4 shows that rocuronium bromide is highly stable during storage in a glass syringe barrel at room temperature, and even at elevated temperatures.

According to another preferred embodiment of the present disclosure, step S2 further comprises sparging the stabilizer solution with an inert gas, preferably after dissolving the at least one stabilizer in the water for injection provided in step S1.

According to another preferred embodiment of the present disclosure, the stabilizer provided in step S2 has an oxygen content of about 2 ppm or less.

Thereby, it is assured that rocuronium bromide or any other component added into said first amount of water for injection cannot react with oxygen, and thus decompose, during the manufacturing of the sterile pharmaceutical composition of rocuronium bromide as shown in example 1.

Step S3: Dissolving a Buffer System in the Stabilizer Solution Obtained from Step S2 to Provide a Buffered Stabilizer Solution

In a third step (S3) of the method according to first aspect of the present disclosure, a buffer system is dissolved in the stabilizer solution obtained from step S2 to provide a buffered stabilizer solution.

Generally, it is understood that the buffer system may comprise any compound that is suitable for adjusting and maintaining the pH of the sterile pharmaceutical composition of rocuronium bromide obtained from step S5 in the desired range.

The inventors of the present disclosure have surprisingly found that it is highly advantageous for the stability of rocuronium bromide in the pharmaceutical composition to dissolve the buffer system after dissolving the at least one stabilizer and before dissolving rocuronium bromide. As can be concluded from Table 3 in Example 1, the pharmaceutical composition only exhibits a minor amount of impurities after preparation, if the at least one stabilizer and the buffer system are dissolved as a second step and third step of the manufacturing method, respectively. Therefore, it is understood that rocuronium bromide is efficiently protected against degradation by dissolving the buffer system in the stabilizer solution obtained from step S2 to provide a buffered stabilizer solution.

According to a preferred embodiment of the present disclosure, the buffer system comprises a pharmaceutically acceptable salt of citric acid, or a pharmaceutically acceptable salt of acetic acid, or a pharmaceutically acceptable salt of glycine, or a mixture thereof. According to another preferred embodiment of the present disclosure, the buffer system comprises a pharmaceutically acceptable salt of citric acid, and a pharmaceutically acceptable salt of acetic acid. According to a further preferred embodiment of the present disclosure, the buffer system is added in an amount so that the buffered rocuronium composition obtained from step S4 comprises sodium citrate·2 H₂O in a concentration of about 5 mg/mL and sodium acetate·3 H₂O in a concentration of about 5 mg/mL.

According to another preferred embodiment of the present disclosure, the pharmaceutically acceptable salt of acetic acid is sodium acetate, such as sodium acetate trihydrate. According to another preferred embodiment of the present disclosure, the pharmaceutically acceptable salt of citric acid is sodium citrate, such as sodium citrate dihydrate. As can be concluded from examples 1, 2, 3 and 4 of the present disclosure, such buffer system is particularly effective in maintaining the pH and protecting rocuronium bromide against chemical decomposition.

According to another preferred embodiment of the present disclosure, the buffer system is added in such amount that the sterile pharmaceutical composition of rocuronium bromide comprised in the prefilled syringe has a pH in the range of from about 3.8 to about 4.2 after storage at 25° C. for up 12 months. According to another preferred embodiment of the present disclosure, the buffer system is added in such amount that the sterile pharmaceutical composition of rocuronium bromide comprised in the prefilled syringe has a pH in the range of from about 3.8 to about 4.0 after storage at 25° C. for 12 months.

It is further possible that also the at least one stabilizer, such as gluconolactone, contributes to the pH of the buffered stabilizer solution obtained in step S3, the buffered rocuronium bromide solution obtained in step S4, and the sterile pharmaceutical composition of rocuronium bromide obtained from step S5.

According to another preferred embodiment of the present, the at least one stabilizer and the buffer system are added in such amount that the buffered rocuronium composition obtained from step S4 has a pH in the range of from about 3.8 to about 4.2. According to a further preferred embodiment of the present wherein the at least one stabilizer and the buffer system are added in such amount that the buffered rocuronium composition obtained from step S4 has a pH in the range of from about 3.8 to about 4.0. It is understood that, if the at least one stabilizer does not have an effect on the pH value, the buffer system is added in such amount that the buffered rocuronium composition obtained from step S4 has a pH in the range of from about 3.8 to about 4.2, or of from about 3.8 to about 4.0.

The inventors of the present disclosure have surprisingly found that the above pH is optimal to ensure good chemical stability of rocuronium bromide in the aqueous solution contained in the glass syringes prepared by the method according to the first aspect of the present disclosure.

However, it is understood that the pH of the final product, i.e. of the sterile pharmaceutical composition of rocuronium bromide obtained from step S5 that is comprised in the prefilled syringe is not adjusted in step S3, but in step S4. To the contrary, the buffered stabilizer solution obtained from step S3 has a pH of at least about 5.5.

The inventors of the present disclosure have surprisingly found that rocuronium bromide is surprisingly stable in aqueous formulations during the manufacturing method when using this sequence of steps (cf. example 1).

In another preferred embodiment of the present disclosure, the pH of the sterile pharmaceutical composition of rocuronium bromide comprised in the prefilled syringe is in the range of from about 3.8 to about 4.2, or in the range of from about 3.8 to about 4.0, after storage for up to 12 months, or up to 24 or 36 months, at room temperature.

According to another preferred embodiment of the present disclosure, step S3 further comprises sparging the buffered stabilizer solution with an inert gas, preferably after dissolving the buffer system in the stabilizer solution provided in step S2.

According to another preferred embodiment of the present disclosure, the stabilizer provided in step S3 has an oxygen content of about 2 ppm or less.

Thereby, it is assured that rocuronium bromide or any other component added into said first amount of water for injection cannot react with oxygen, and thus decompose, during the manufacturing of the sterile pharmaceutical composition of rocuronium bromide as shown in example 1.

Step S4: Dissolving Rocuronium Bromide in the Buffered Stabilizer Solution Obtained from Step S3

In a fourth step (S4) of the method according to first aspect of the present disclosure, rocuronium bromide is dissolved in the buffered stabilizer solution obtained from step S3 and water for injection is added in an amount to provide a buffered rocuronium bromide solution, wherein the buffered rocuronium composition comprises rocuronium in an amount of about 10 mg/mL.

Rocuronium bromide is added in an amount so that the buffered rocuronium composition comprises rocuronium in an amount of about 10 mg/mL, which is the standard concentration of rocuronium bromide in the clinical practice.

According to a preferred embodiment of the present disclosure, step S4 comprises mixing the buffered rocuronium composition for at least 10 h until a stable pH is obtained. According to a preferred embodiment of the present disclosure, step S4 comprises mixing the buffered rocuronium composition for at least 20 h until a stable pH is obtained. According to another preferred embodiment of the present disclosure, step S4 comprises mixing the buffered rocuronium composition for at least 30 h until a stable pH is obtained.

The inventors of the present disclosure have surprisingly found that the pH of the solution obtained in step S4 is subject to a slow pH adjustment upon stirring as shown in the FIGURE of the present disclosure: After addition of the rocuronium bromide and water for injection, the pH of the resulting solution has a pH well above 5.0. Within about 10 h of stirring, the pH drops to about 4.0 and stabilizes between 4.0 and 3.8 without the addition of any further pH adjusting agent. In other words, the pH changes automatically, without being actively adjusted, i.e., without the addition of an acid. This pH shift is highly surprising and could potentially have detrimental effects on the product stability and patient safety, if this pH shift is overlooked or not taken into account.

However, the inventors of the present disclosure have surprisingly found that both the sequence of steps and the slow adaption of the pH value have a positive effect on the stability of rocuronium bromide during manufacturing of the prefilled syringes:

As can be concluded from Table 3 of Example 1, the pharmaceutical composition only exhibits a minor amount of impurities after preparation, if the rocuronium bromide is dissolved in the buffered stabilizer solution obtained from step S3. Therefore, it is understood that rocuronium bromide is efficiently protected against degradation by dissolving rocuronium bromide in the buffered stabilizer solution obtained from step S3 to provide a buffered rocuronium composition.

Furthermore and without wishing to be bound by theory, the slow pH shift during stirring ensures that the rocuronium bromide is dissolved in an aqueous solution with almost neutral pH which is only slowly adjusted to about 3.8 to 4.2 or 3.8 to 4.0. As no acid is added during this pH adjustment, rocuronium bromide is highly stable and is—in particular—not hydrolyzed by the acid (cf. Table 3 of Example 1).

According to another preferred embodiment of the present disclosure, the buffered rocuronium composition obtained from step S4 has a pH in the range of from about 3.8 to about 4.2. According to a further preferred embodiment of the present disclosure, the buffered rocuronium composition obtained from step S4 has a pH in the range of from about 3.8 to about 4.0.

The inventors of the present disclosure have surprisingly found that this pH stabilizes the rocuronium bromide in aqueous solution thus providing for a good shelf-life of the prefilled syringes prepared by the method according to the first aspect of the present disclosure. As can be concluded from example 4 of the present disclosure, rocuronium bromide is highly stable and not susceptible to chemical degradation during storage at room temperature, even during storage at elevated temperature. At the same time, the sterile rocuronium composition has good tolerability and does not provoke injection site reactions, such as pain and/or itching, upon injection to a patient.

According to another preferred embodiment of the present disclosure, the at least one stabilizer is gluconolactone and the buffered rocuronium composition obtained from step S4 comprises gluconolactone in an amount of about 25 mg/mL. The inventors of the present disclosure have surprisingly found that such concentration of gluconolactone particularly stabilizes the rocuronium bromide in aqueous solution during manufacturing (cf. examples 1 and 2), sterilization (cf. example 3), and storage (cf. example 4). Therefore, rocuronium bromide is highly stable and not susceptible to chemical degradation during storage at room temperature, even during storage at elevated temperature. Hence, the shelf-life of a prefilled syringe prepared by the method according to the first aspect of the present disclosure is prolonged.

Furthermore, it may be assumed that gluconolactone helps to slowly adjust the pH during step S4, especially during stirring for at least about 10 h, or at least about 20 h, or at least about 30 h.

According to another preferred embodiment of the present disclosure, the buffered rocuronium composition obtained from step S4 comprises sodium citrate·2 H₂O in a concentration of about 5 mg/mL and sodium acetate·3 H₂O in a concentration of about 5 mg/mL.

The inventors of the present disclosure have surprisingly found that this buffer system has a particularly positive effect on the stability of rocuronium bromide in aqueous solution during manufacturing (cf. examples 1 and 2), sterilization (cf. example 3), and storage (cf. example 4). Therefore, rocuronium bromide is highly stable and not susceptible to chemical degradation during storage at room temperature, even during storage at elevated temperature. Hence, the shelf-life of a prefilled syringe prepared by the method according to the first aspect of the present disclosure is prolonged.

As can be concluded from example 4 of the present disclosure, rocuronium bromide is highly stable and not susceptible to chemical degradation during storage at room temperature, even during storage at elevated temperature. The pH does not shift significantly.

According to another preferred embodiment of the present disclosure, the buffered rocuronium formulation obtained from step S4 has an osmolarity in the range of from about 270 mOsmol/kg to about 310 mOsmol/kg. This osmolarity ensures that the buffered rocuronium formulation or the sterile pharmaceutical composition of rocuronium bromide obtained in step S5 is ready to inject without further dilution. Thus, the pharmaceutical composition of rocuronium bromide has good tolerability and does not provoke injection site reactions, such as pain and/or itching, upon injection to a patient.

If necessary, the osmolarity may be adjusted during any one of steps S2, S3, or S4 by adding a tonicity agent. The tonicity agent may be selected from the group consisting of dextrose, mannitol, potassium chloride, and sodium chloride. The tonicity agent may preferably be sodium chloride. Therefore, in another preferred embodiment of the present disclosure, any one of steps S2, S3, or S4 further comprises adding a tonicity agent.

In some embodiments, the tonicity agent is present in an amount sufficient to make the composition obtained from step S4 isotonic. In some embodiments, the tonicity agent is present in the buffered rocuronium composition in an amount sufficient to provide an osmolality in the range of from about 270 mOsm/kg to about 310 mOsm/kg.

In another embodiment of the present disclosure, the sterile pharmaceutical composition of rocuronium bromide in the prefilled syringe has an osmolality in the range of from about 270 mOsm/kg to about 310 mOsm/kg. It is understood that said osmolarity does not change over time. In another embodiment of the present disclosure, the sterile pharmaceutical composition of rocuronium bromide in the prefilled syringe has an osmolality in the range of from about 270 mOsm/kg to about 310 mOsm/kg, after storage for up to 12 months, or up to 24 or 36 months, at room temperature.

According to another preferred embodiment of the present disclosure, the buffered rocuronium formulation obtained from step S4 has a titratable acidity of at least about 60 mEq. According to another preferred embodiment of the present disclosure, the buffered rocuronium formulation obtained from step S4 has a titratable acidity of at least about 80 mEq. According to another preferred embodiment of the present disclosure, the buffered rocuronium formulation obtained from step S4 has a titratable acidity of at least about 100 mEq. According to another preferred embodiment of the present disclosure, the buffered rocuronium formulation obtained from step S4 has a titratable acidity in the range of from about 100 mEq to about 150 mEq.

Titratable acidity within this range contributes to the stability of rocuronium bromide in the prefilled syringe during storage (cf. example 4) and prevents pH-dependent degradation due to pH shifts. In other words, maintaining a specific pH helps preserve the chemical integrity and potency of rocuronium bromide over time, and at the same time minimizes irritation or discomfort upon injection.

According to another preferred embodiment of the present disclosure, step S4 further comprises sparging the buffered stabilizer solution buffered rocuronium bromide solution, preferably after dissolving the rocuronium bromide and adding water for injection to the buffered stabilizer solution provided in step S3.

According to another preferred embodiment of the present disclosure, the stabilizer provided in step S4 has an oxygen content of about 2 ppm or less.

Thereby, it is assured that rocuronium bromide or any other component added into said first amount of water for injection cannot react with oxygen, and thus decompose, during the manufacturing of the sterile pharmaceutical composition of rocuronium bromide as shown in example 1.

Step S5: Sterilizing the Buffered Rocuronium Composition Obtained from Step S4

In a fifth step (S5) of the method according to the first aspect of the present disclosure, the buffered rocuronium composition obtained from step S4 is sterilized to provide a sterile pharmaceutical composition of rocuronium bromide.

Sterilizing the buffered rocuronium composition obtained from step S5 to provide a sterile pharmaceutical composition of rocuronium bromide can be performed by any method known to the skilled person.

According to a preferred embodiment of the present disclosure, sterilization is performed by terminal sterilization or sterile filtration. According to a further preferred embodiment of the present disclosure, sterilization is performed by terminal sterilization.

It is understood that, when sterilization is performed by terminal sterilization, the order of steps S5 and S6 may be carried out in reverse order. In other words, according to a preferred embodiment of the present disclosure, the buffered rocuronium composition obtained from step S4 is filled into a glass syringe to provide a glass syringe comprising the buffered rocuronium composition, and subsequently said glass syringe comprising the buffered rocuronium composition is sterilized by terminal sterilization to provide a glass syringe comprising the sterile pharmaceutical composition of rocuronium bromide.

According to a preferred embodiment of the present disclosure, step S5 is a terminal sterilization at about 121° C. for a period F₀ of about 8 minutes. According to another preferred embodiment of the present disclosure, step S5 is a terminal sterilization at about 121° C. for a period F₀ of about 15 minutes. According to another preferred embodiment of the present disclosure, step S5 is a terminal sterilization at about 121° C. for a period F₀ of about 20 minutes.

The inventors of the present disclosure have surprisingly found that the rocuronium is efficiently stabilized during terminal sterilization. To the contrary, terminal sterilization merely results in the formation of minor amounts of impurity C in the range of from 0.3% to 0.5% despite the high thermal stress (cf. Table 4).

According to a further preferred embodiment of the present disclosure, step S5 is performed by sterile filtration. Sterile filtration may be achieved by filtering the buffered rocuronium composition obtained from step S4 through one or more, preferably two, filters having a maximum pore size of about 0.2 μm.

It is understood that the filter must be made of a material that is compatible with the buffered rocuronium composition obtained from step S4. In other words, the filter must be made of a material that does neither react with any component of the buffered rocuronium composition obtained from step S4 nor adsorbs any one of the components of the buffered rocuronium composition obtained from step S4. A non-limiting example for such a filter material is polyethersulfone (PES).

A non-limiting example of a filter is Sartopore® 2 (obtained from Sartorius Stedim, Goettingen, Germany or Sartorious Stedim North America, Inc., Bohemia, NY, USA).

The inventors have surprisingly found that sterile filtration provides for the highest stability of rocuronium bromide during sterilization. As can be concluded from Table 4, the amount of impurity C does not change during filtration, while also the amount of rocuronium bromide is not significantly different before and after filtration.

Therefore, sterile filtration is the most preferable method for sterilizing the buffered rocuronium composition obtained from step S4 to obtain a sterile pharmaceutical composition of rocuronium bromide.

Step S6: Filling the Sterile Pharmaceutical Composition of Rocuronium Bromide Obtained from Step S5 into a Glass Syringe Under Aseptic Conditions

In a sixths step (S6) of the method according to the first aspect of the present disclosure, the sterile pharmaceutical composition of rocuronium bromide obtained from step S5 is filled into a glass syringe under aseptic conditions to provide a glass syringe comprising the sterile pharmaceutical composition of rocuronium bromide.

Procedures for filling the sterile pharmaceutical composition of rocuronium bromide into a syringe, and their subsequent processing, such as aseptic filling conditions, are known in the art. In one embodiment, the sterile pharmaceutical composition of rocuronium bromide is aseptically filled into the glass syringe.

According to a preferred embodiment of the present disclosure, in step S6, the volume of the sterile pharmaceutical composition of rocuronium bromide obtained from step S5 is adjusted in that the deliverable volume of the sterile pharmaceutical composition of rocuronium bromide is in the range of from about 5 mL to about 10 mL, preferably about 5 mL, wherein the deliverable volume is determined according to the USP General chapter <657> as in force on 25 Mar. 2024.

By that, it is ensured that the dose of rocuronium bromide can be delivered to the patient in an accurate manner.

According to another preferred embodiment, the glass syringe is sterile. Methods for sterilizing a glass syringe are known in the art. Thus, sterility of all components can be maintained.

According to a preferred embodiment of the present disclosure, step S6 is carried out in a filling machine, wherein said filling machine is preferably located in a grade A cleanroom. The filling machine carries out the step of accurately filling containers with the pharmaceutical composition of rocuronium bromide. According to another preferred embodiment of the present disclosure, the filling machine is equipped with a manometer that is adapted to measure and control the pressure inside and outside the filling machine. According to another preferred embodiment of the present disclosure, the pressure in the filling machine is set and maintained at 4% of the atmospheric pressure in the grade A cleanroom or less, preferably 4%.

According to a preferred embodiment of the present disclosure, step S6 is carried out under an atmosphere of nitrogen and up to about 4% (V/V) of oxygen. According to another preferred embodiment of the present disclosure, step S6 is carried out under an atmosphere of nitrogen and of from 0.2% to about 4% (V/V) of oxygen. According to another preferred embodiment of the present disclosure, step S6 is carried out under an atmosphere of nitrogen and of from 0.5% to about 4% (V/V) of oxygen. According to another preferred embodiment of the present disclosure, step S6 is carried out under an atmosphere of nitrogen and of from 1% to about 4% (V/V) of oxygen. According to another preferred embodiment of the present disclosure, step S6 is carried out under an atmosphere of nitrogen and of from 1.5% to about 4% (V/V) of oxygen. According to another preferred embodiment of the present disclosure, step S6 is carried out under an atmosphere of nitrogen and of from 2% to about 4% (V/V) of oxygen.

Therefore, it is understood that the headspace in a prefilled glass syringe prepared by the method according to the first aspect of the present disclosure, that is a space filled with gas that is present in the glass syringe barrel together with the sterile pharmaceutical composition of rocuronium bromide, contains the above disclosed concentration of oxygen.

The inventors of the present disclosure have surprisingly found that it is not necessary to carry out the process under strict exclusion of oxygen. This makes the manufacturing process more convenient and less cost intensive, i.e. cheaper. Generally, the demands for the manufacturing facilities are lowered. At the same time, the stability of rocuronium bromide in the prefilled glass syringes prepared according to the method of the first aspect of the present disclosure is not affected: To the contrary, it is shown in example 4 of the present disclosure that the rocuronium bromide is highly stable during storage in a glass syringe barrel at room temperature, and even at elevated temperatures.

Step S7: Blanketing the Glass Syringe Comprising the Sterile Pharmaceutical Composition of Rocuronium Bromide Obtained from Step S6

In a seventh step (S7) of the method according to the first aspect of the present disclosure, the glass syringe comprising the sterile pharmaceutical composition of rocuronium bromide obtained from step S6 is blanketed with an inert gas and a plunger is inserted into said glass syringe to provide the prefilled glass syringe comprising the sterile pharmaceutical composition of rocuronium bromide.

According to a preferred embodiment of the present disclosure, step S7 is carried out under an atmosphere of nitrogen and up to about 4% (V/V) of oxygen. According to another preferred embodiment of the present disclosure, step S7 is carried out under an atmosphere of nitrogen and of from 0.2% to about 4% (V/V) of oxygen. According to another preferred embodiment of the present disclosure, step S7 is carried out under an atmosphere of nitrogen and of from 0.5% to about 4% (V/V) of oxygen. According to another preferred embodiment of the present disclosure, step S7 is carried out under an atmosphere of nitrogen and of from 1% to about 4% (V/V) of oxygen. According to another preferred embodiment of the present disclosure, step S7 is carried out under an atmosphere of nitrogen and of from 1.5% to about 4% (V/V) of oxygen. According to another preferred embodiment of the present disclosure, step S7 is carried out under an atmosphere of nitrogen and of from 2% to about 4% (V/V) of oxygen.

Therefore, it is understood that the headspace in a prefilled glass syringe prepared by the method according to the first aspect of the present disclosure, that is a space filled with gas that is present in the glass syringe barrel together with the sterile pharmaceutical composition of rocuronium bromide, contains the above disclosed concentration of oxygen.

The inventors of the present disclosure have surprisingly found that it is not necessary to carry out the process under strict exclusion of oxygen. This makes the manufacturing process more convenient and less cost intensive, i.e. cheaper. Generally, the demands for the manufacturing facilities are lowered. At the same time, the stability of rocuronium bromide in the prefilled glass syringes prepared according to the method of the first aspect of the present disclosure is not affected: To the contrary, it is shown in example 4 of the present disclosure that the rocuronium bromide is highly stable during storage in a glass syringe barrel at room temperature, and even at elevated temperatures.

According to a preferred embodiment of the present disclosure, the sterile pharmaceutical composition of rocuronium bromide comprised in the prefilled syringe obtained from step S7 comprises at most 0.2% of total impurities, as determined by HPLC.

It is advantageous that the sterile pharmaceutical composition of rocuronium bromide comprised in the prefilled syringe obtained from step S7 contains a minimum amount of impurities. This increases the shelf-life of the prefilled glass syringes prepared by the method according to the first aspect of the present disclosure.

The inventors of the present disclosure have surprisingly found that the specific sequence of steps S1, S2, S3, S4, S5, S6, and S7 enables the manufacture of prefilled syringes comprising a sterile pharmaceutical composition of rocuronium bromide comprised in the prefilled syringe obtained from step S7 comprises at most 0.2% of total impurities, as determined by HPLC, as shown in examples 1 to 3.

### General

According to a preferred embodiment of the present disclosure, the glass syringe prepared by the method according to the present disclosure has a glass syringe barrel, a rubber stopper, a plunger rod, and a Luer Lock.

The glass syringe barrel is adapted to take up the sterile pharmaceutical composition of rocuronium bromide obtained from step S5. Thus, the sterile pharmaceutical composition of rocuronium bromide is in direct contact with the glass surface of the glass syringe barrel. Glass is not susceptible to adsorption of rocuronium bromide or any other component of the sterile pharmaceutical composition of rocuronium bromide. As can be concluded from example 4 of the present disclosure, rocuronium bromide is highly stable during storage in a glass syringe barrel at room temperature, and even at elevated temperatures.

The Luer Lock improves the handling of the glass syringe prepared by the method according to the first aspect of the present disclosure. Thus, the sterile pharmaceutical composition of rocuronium bromide can be conveniently administered to a patient from the glass syringe.

The method of the first aspect of the present disclosure yields a prefilled syringe that comprises a sterile pharmaceutical composition of rocuronium bromide. Here, “sterile pharmaceutical composition of rocuronium bromide” means an aqueous formulation that is suitable for administration to a patient, e.g. by intravenous injection, comprising rocuronium bromide, at least one stabilizer as disclosed herein, a buffer system as disclosed herein, optionally a tonicity agent as disclosed herein, and water for injection.

In the method according to the first aspect of the present disclosure, the sterile pharmaceutical composition of rocuronium bromide may be prepared in a bulk and filled into the syringe in step S6.

According to another preferred embodiment of the present disclosure, the sterile pharmaceutical composition of rocuronium bromide comprised in the prefilled syringe contains at least 9.8 mg/mL of rocuronium bromide after storage at 25° C. for up to 6 months, as determined by HPLC.

According to another preferred embodiment of the present disclosure, wherein the sterile pharmaceutical composition of rocuronium bromide comprised in the prefilled syringe contains at most about 1.3% of total impurities after storage at 25° C. for up to 6 months, as determined by HPLC.

The inventors of the present disclosure have surprisingly found that the prefilled syringes prepared by the method according to the first aspect of the present disclosure have a prolonged shelf-life of at least 6 months. Example 4 shows that rocuronium bromide is highly stable during storage in a glass syringe barrel at room temperature, and even at elevated temperatures.

According to a preferred embodiment of the present disclosure, any one of steps S2, S3, S4, and/or S5 further comprise sparging with nitrogen after each addition of compound or during said step.

### The Prefilled Syringe Prepared by the Method According to the First Aspect of the Present Disclosure

According to a second aspect, the present disclosure relates to a prefilled glass syringe comprising a sterile pharmaceutical composition of rocuronium bromide obtained by the method according to the first aspect of the present disclosure.

The sterile pharmaceutical composition of rocuronium bromide comprised in the prefilled syringes of the present disclosure can be used for intravenous injection in a bolus dose without dilution.

Therefore, the prefilled glass syringe according to the second aspect of the present disclosure can be conveniently handled by medical staff without the danger of cross contaminations or the like.

Furthermore, the prefilled glass syringe according to the second aspect of the present disclosure can be stored at room temperature as shown in example 4. In other words, the prefilled glass syringe according to the second aspect of the present disclosure do not require refrigeration.

### The Kit Comprising a Prefilled Syringe Prepared by the Method According to the First Aspect of the Present Disclosure or a Prefilled Syringe According to the Second Aspect of the Present Disclosure

According to a third aspect, the present disclosure relates to a kit comprising
i) the prefilled glass syringe comprising a sterile pharmaceutical composition of rocuronium bromide according the second aspect of the present disclosure or prepared by the method according to the first aspect of the present disclosure,
ii) a plunger rod, and
iii) a carton box as secondary packaging.

The prefilled syringes containing the rocuronium bromide solution can be provided as a kit, where it is packaged in a clear, flexible plastic overwrap, or the like.

The overwrap protects the prefilled syringes from physical damage resulting from shipping and/or handling, and also provides evidence of potential product tampering, for example, if the overwrap is open or has a slit or tear in it.

### Definitions and General Embodiments

As used herein, the term “rocuronium” refers to a cation of the name [3-hydroxy-10,13-dimethyl-2-morpholin-4-yl-16-(1-prop-2-enyl-2,3,4,5-tetrahydropyrrol-1-yl)-2,3,4,5,6,7,8,9,11,12,14,15,16,17-tetradecahydro-1H-cyclopenta[a]phenanthren-17-yl]acetate. In other words, the term “rocuronium” as used herein refers to a compound of the structure

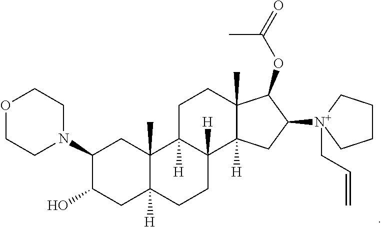

It is understood that rocuronium is a cation.

As used herein, the term “rocuronium bromide” refers to a salt consisting of rocuronium as the cation and bromide (Br) as the anion. Rocuronium bromide is also known under CAS-number 119302-91-9.

It is further understood that rocuronium is susceptible to degradation, especially when stored in aqueous formulations. A major degradation product is formed through acid catalyzed or base mediated hydrolysis of the acetic ester group to form a compound, which is also referred to as “impurity C” herein, of formula

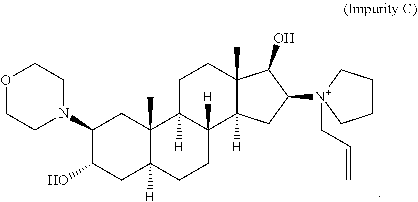

Another degradation product is formed through oxidation to form a compound, which is also referred to as “impurity H” herein, of formula

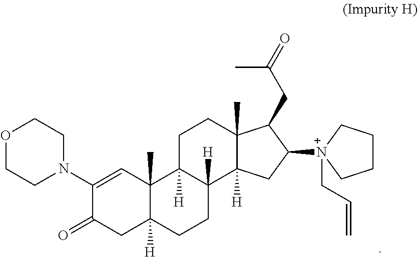

As used herein, the term “room temperature” refers to a temperature of about 25° C.

As used herein, the temperature T at which sterilization is performed is the temperature of the container.

As used herein, when sterilization is performed for a specific period, said period refers to the F₀ value. F₀ is a quantitative parameter used to evaluate the amount of heat transferred to a product or solution during a heat sterilization process, and describes the lethality performance of the process.

According to one embodiment, F₀ can be calculated through the following formula:

$${F}_{0} = {\int }_{0}^{t} 1 {0}^{\frac{T - 121 ° C .}{Z}} dt$$

wherein Z is 10° C., and T is the temperature of the container at time point t. This formula can be applied when the temperature T of the container changes during sterilization.

In practice, the sterilizer, such as an autoclave or water cascade sterilizer, heats the product (e.g., container) for a certain period of time until the sterilization temperature of, e.g., 121° C. is reached. During the heating time for heating from 100° C. to 121° C., and also during the cooling time, microorganisms are killed. Taking this killing effect during the heating and cooling time into account, the holding time can be shortened accordingly. Thus, the desired F₀ value can be achieved with a shorter overall process time.

In other words, the F₀ value of a terminal sterilization process is a measure for the lethality, expressed in terms of the equivalent time in minutes at a temperature T of 121° C. delivered by the process to the load in its container with reference to micro-organisms possessing a theoretical Z-value of 10° C. For instance, an F₀ value of 1 min is the heat effect (lethal effect) of 121° C. within one minute.

According to another embodiment, the period F₀ for which sterilization is performed, is calculated on basis of the time t for which the container is held at a constant temperature T. If the container is held at a constant temperature T for a total time t during sterilization, the above formula simplifies as follows:

$${F}_{0} = t \cdot {10}^{\frac{T - 121 ° C .}{Z}}$$

wherein Z is 10° C., T is the temperature of the container, and t is the time for which the container is held at temperature T.

Furthermore, it the container is held at a temperature T of 112° C. for 15 min, the above equation simplifies to

$${F}_{0} = 15 min \cdot {10}^{\frac{112 ° C . - 121 ° C .}{10 ° C .}} = 15 min \cdot {10}^{- 0.9} \approx 1.9 min$$

In other words, a 15 min sterilization at 112° C. is equivalent, in terms of lethal effect, to about 1.9 min at 121° C.

The term “about” in conjunction with a numerical value refers to normal deviations of said numerical value. It is to be understood that the term “about” can mean a deviation of +10%, preferably +5%, more preferably +2.5% of said numeric value as indicated.

As used herein, the term “sparging” refers to removing oxygen inside a liquid, such as any one of the solutions obtained from S1, S2, S3, S4, and/S5, by actively bubbling an inert gas, preferably nitrogen, through said liquid. According to a preferred embodiment of the present disclosure, sparging is performed for at least 15 min. According to a preferred embodiment of the present disclosure, sparging is performed at a reduced pressure, preferably at about 5 psi (about 345 mbar).

As used herein, the term “blanketing” refers to overlaying the sterile pharmaceutical composition comprised in the glass syringe, e.g. in the glass syringe comprising the sterile pharmaceutical composition of rocuronium bromide obtained from step S6, with a blanket of inert gas, such as nitrogen, in the overhead space. In a preferred embodiment of the present disclosure, blanketing is carried the inert gas contains up to about 4% (V/V) of oxygen, such as of from about 0.5% (V/V) to about 4% (V/V), or of from about 1% (V/V) to about 4% (V/V), or of from about 1.5% (V/V) to about 4% (V/V), or of from about 2% (V/V) to about 4% (V/V). According to another preferred embodiment of the present disclosure, blanketing is performed at an increased pressure, such as at least about 1 atm (about 1013 mbar), or at about 20 psi (about 1,379 mbar). The inert gas used for blanketing forms the gas in the headspace in the prefilled syringe.

As used herein, an “inert gas” is a gas that does not readily undergo chemical reactions with other chemical substances and therefore does not readily form chemical compounds. Preferred inert gases are argon or nitrogen. Nitrogen is a particularly preferred inert gas. It is understood that nitrogen is advantageous as it is readily available and cheap, but ensures sufficient stability of rocuronium bromide during storage of the prefilled syringe as shown in example 4.

As used herein, the term “water for injection” refers water that is suitable for injection. In a preferred embodiment of the present disclosure, “water for injection” fulfills the respective requirements of the USP and/or the European Pharmacopoeiea as in force on 25 Mar. 2024. Water for injection is commercially available. A non-limiting example of water for injection is the commercial product marked under the registered trademark TITAN XL® Container (Pharmacy Bulk Pack) with product code S8506 (obtainable from B. Braun Medical Inc., Bethlehem, PA, USA).

As used herein, the term “sterile pharmaceutical composition of rocuronium bromide” refers to a formulation of rocuronium bromide that is injectable.

As used herein, the term “headspace” refers to the gas-filled space between the plunger and the sterile pharmaceutical composition of rocuronium bromide within the syringe barrel. This space exists to accommodate changes in volume that may occur due to factors such as temperature variations, pressure changes, or movement of the plunger during shipping and handling. Furthermore, the presence of the headspace ensures that the deliverable volume as disclosed hereinabove is achieved. It is understood that the composition of the gas in the headspace may be important for the stability of rocuronium bromide during storage.

As used herein, the “oxygen content” of a liquid, such as the oxygen content of the first amount of water for injection provided in step S1, is determined with a calibrated oxygen sensor. Preferably, said calibrated oxygen sensor is calibrated by creating a calibration curve by measuring a plurality of approved and commercially available standards having an oxygen content in the range of from 0% to 100% (saturated with oxygen).

As used herein, the “oxygen content” of a gas, such as the oxygen content of the atmosphere in step S6 and/or the oxygen concentration in the headspace is determined with an oxygen analyzer, such as a FMS-Oxygen Headspace Analyzer (Lighthouse Instruments, LLC; Charlottesville; VA; USA).

As used herein, the “titratable acidity” refers to the quantity (in mEq) of sodium hydroxide consumed in the titration of 1 L of the buffered rocuronium formulation obtained from step S4 to pH 7.4. As used herein, the titratable acidity is determined by titration, preferably at room temperature.

### Assay (1)—Determination of the Amount of Rocuronium Bromide

For the determination of the amount of rocuronium bromide comprised in a pharmaceutical composition, a 15 min liquid chromatography method is used.

For the assay, a sample of 5 μL of the pharmaceutical composition comprising rocuronium bromide is analyzed on a Agilent HPLC system equipped with a variable wavelength detector (VWD) or a diode array detector (DAD) using a Phenomonex Kinetex Hilic column (150×4.6 mm, 2.6 μm; part no. 00F-4461-E0) maintained at 30° C. and at a flow rate of 1.80 mL/min. Elution conditions involve an isocratic elution with a mobile phase consisting of 89% (V/V) of acetonitrile and 11% (V/V) of a tetramethylammonium hydroxide buffer (25 mM; adjusted to pH 6.60).

Rocuronium is detected by UV detection at 210 μm and the peak area is calculated.

The amount of rocuronium in %, such as % (w/w), is performed by external calibration using a working standard of rocuronium whose purity assay was determined at each time point by a mass balance approach (100%-sum of impurities).

### Assay (II)—Determination of the Amount of Impurities

For the determination of the amount of impurities comprised in a pharmaceutical composition, a 15 min liquid chromatography method is used.

For the assay, a sample of 20 μL of the pharmaceutical composition comprising rocuronium bromide is analyzed on a Agilent HPLC system equipped with a variable wavelength detector (VWD) or a diode array detector (DAD) using a Phenomonex Kinetex Hilic column (150×4.6 mm, 2.6 μm; part no. 00F-4461-E0) maintained at 30° C. and at a flow rate of 1.80 mL/min. Elution conditions involve an isocratic elution with a mobile phase consisting of 89% (V/V) of acetonitrile and 11% (V/V) of a tetramethylammonium hydroxide buffer (25 mM; adjusted to pH 6.60).

Impurities are detected by UV detection at 210 μm and the respective peak areas of each impurity are calculated.

The amount of impurities in %, such as % (w/w), is performed by external calibration (working standard of rocuronium), taking into account the relative response factor (RRF), sample dilution and Assay. The amount of impurities (% Impurity) is determined according to the following formula:

$$\% Impurity = \frac{area sample * std conc . * 10 mL * R R F * 100}{area Std * 1. mL * assay}$$

Where: “area sample” is the peak area of the respective impurity determined from the chromatogram when measuring the pharmaceutical composition comprising rocuronium bromide with assay (II), “area Std” is the average peak area of rocuronium determined from the chromatogram when measuring a rocuronium working standard solution (n=6) with assay (II), “std conc.” is the concentration of rocuronium in said rocuronium working standard solution in mg/mL, “assay” is the concentration of rocuronium bromide in the sample determined according to assay (I) (such as 10 mg/mL) or is set to 10 mg/mL, and RRF is the relative response factor of the respective impurity. As used herein, the RRF of an impurity is obtained from the ratio between the slope of the linearity curve of the impurity against the slope of the linearity curve of the drug (rocuronium). This value may be calculated during the validation of the analytical method in the linearity parameter. Said RRF must be calculated for each of the known impurities to be quantified during the analysis of impurities in the pharmaceutical compositions as disclosed herein. In other words, the RRF of each impurity may be determined according to the following equation:

$$R R F = \frac{slope of impurity peak}{slope of rocuronium peak}$$

wherein “slope of impurity peak” is the slope of the peak recorded for an impurity determined by linear regression analysis or by calculating the rise of signal for two points on the chromatogram, and “slope of rocuronium peak” is the slope of the peak recorded for rocuronium determined by linear regression analysis or by calculating the rise of signal for two points on the chromatogram.

### EXAMPLES

### Example 1: Preparation of Pharmaceutical Composition Comprising Rocuronium Bromide

To investigate the influence of the order of addition of the components during manufacturing of a pharmaceutical composition comprising rocuronium bromide on the chemical stability of rocuronium bromide in said pharmaceutical composition, the method of manufacturing was conceptually divided into the following steps S1, S2, S3, S4, and Sf:
S1: Providing a first amount of water for injection, wherein said first amount of water corresponds to 50% (V/V) of the total volume of the pharmaceutical composition comprising rocuronium bromide to be prepared.
S2: Dissolving gluconolactone as the at least one stabilizer in the product obtained from the respective previous step, wherein the amount of gluconolactone is chosen so that the concentration of gluconolactone in the total volume of the pharmaceutical composition of rocuronium bromide is 25 mg/mL.
S3: Subsequently dissolving sodium citrate dihydrate and sodium acetate trihydrate in the product obtained in the respective previous step, wherein
   a) the amount of sodium citrate dihydrate is chosen so that the concentration of sodium citrate dihydrate in the total volume of the pharmaceutical composition of rocuronium bromide is 5 mg/mL, and
   b) the amount of sodium acetate trihydrate is chosen so that the concentration of sodium acetate trihydrate in the total volume of the pharmaceutical composition of rocuronium bromide is 5 mg/mL.
S4: Dissolving rocuronium bromide in the product obtained from the respective previous step, wherein the amount of rocuronium bromide is chosen so that the concentration of rocuronium bromide in the total volume of the pharmaceutical composition of rocuronium bromide is 10 mg/mL.
Sf: Adding a second amount of water for injection that is required to prepare the total volume of the pharmaceutical composition comprising rocuronium bromide.

Pharmaceutical compositions comprising rocuronium bromide were manufactured by different methods (see Table 2), each method yielding a pharmaceutical composition having the composition shown in Table 1.

TABLE 1
Composition of the pharmaceutical composition
comprising rocuronium bromide

| Component | Function | Concentration | |
|---|---|---|---|
| Rocuronium bromide | Active pharmaceutical | 10 | mg/mL |
| | ingredient | | |
| Sodium citrate · 2 H₂O | Buffer system | 5 | mg/mL |
| Sodium acetate · 3 H₂O | | 5 | mg/mL |
| Gluconolactone | Stabilizer | 25 | mg/mL |

| | | |
|---|---|---|
| Water for injection | Solvent | q.s. to final |
| | | vol. |

The method for manufacturing a pharmaceutical composition according to Table 1 were modified in that the order of steps S2, S3, and S4 was changed as shown in Table 2:

TABLE 2
Method of manufacturing of a pharmaceutical composition comprising
rocuronium. Method steps S1, S2, S3, S4, and Sf were carried
out subsequently in the order from top to bottom.

| | | method | method | method | method | method |
|---|---|---|---|---|---|---|
| Order of | method | 2 | 3 | 4 | 5 | 6 |
| steps | 1 | (comp.) | (comp.) | (comp.) | (comp.) | (comp.) |
| 1^{st} step | S1 | | | | | |
| 2^{nd} step | S2 | S4 | S2 | S4 | S3 | S3 |
| 3^{rd} step | S3 | S3 | S4 | S2 | S2 | S4 |
| 4^{th} step | S4 | S2 | S3 | S3 | S4 | S2 |

| | |
|---|---|
| 5^{th} step | Sf |

Comp.: Comparative

The compositions prepared by the methods according to Table 2 were analyzed for the amount of rocuronium bromide according to Assay (I) and amount of impurities according to Assay (II) to evaluate the chemical stability of rocuronium bromide during manufacturing. Results are shown in Table 3.

TABLE 3
Results of stability studies of rocuronium bromide during manufacturing

| | Rocuronium bromide composition according to Table 1 prepared by | | | | | |
|---|---|---|---|---|---|---|
| | | method 2 | method 3 | method 4 | method 5 | method 6 |
| | method 1 | (comp.) | (comp.) | (comp.) | (comp.) | (comp.) |
| Rocuronium | 103.6 | 103.6 | 100.9 | 101.5 | 102.7 | 100.7 |
| bromide [%] | | | | | | |
| Impurity A [%] | <LoQ | <LoQ | <LoQ | <LoQ | <LoQ | <LoQ |
| Impurity H [%] | <LoQ | <LoQ | <LoQ | n.d. | n.d. | n.d. |
| Impurity C [%] | 0.1 | 0.6 | 0.1 | 0.3 | 0.2 | 0.4 |

LoQ: Limit of quantification
n.d.: Not determined

The results shown in Table 3 indicate that method 1 as disclosed in Table 2 yields a pharmaceutical composition with the least impurities and the highest amount of rocuronium bromide. On the other hand, adding the rocuronium bromide before adding at least one stabilizer (i.e., gluconolactone; cf. Table 3, results obtained for compositions prepared by methods 2, 4, and 6) results in a considerably increased formation of impurities and decomposition of rocuronium bromide.

In view of the above, the pharmaceutical composition prepared according to method 1, i.e. subsequently dissolving the at least one stabilizer (S2), dissolving the buffer system (S3), and then dissolving rocuronium bromide (S4), was selected for further investigation.

### Example 2: PH Adjustment and pH Stabilization

A pharmaceutical composition comprising rocuronium bromide was prepared according to example 1, method 1. After preparation, said formulation was held at room temperature for more than 90 h and the pH was measured at several time points. Results of the pH measurement are shown in the FIGURE.

As can be concluded from the FIGURE, the pH of the composition is well above 5 directly after preparation. However, the pH decreases over time without the addition of HCl. After about 20 h, the pH automatically stabilizes in the desired range of about 3.8 to 4.0.

It is understood that the pH of a pharmaceutical composition is critical for both the stability of rocuronium bromide, as well as the occurrence of injection site reactions upon intravenous injection, such as pain and/or itching.

The inventors of the present disclosure have surprisingly found that the pharmaceutical composition comprising rocuronium bromide exhibit a pH of about 3.8 to about 4.0 even without adjustment of pH with HCl and/or NaOH. To the contrary, it was surprisingly found that the pH of the pharmaceutical composition is automatically adjusted to about 3.8 to about 4.0 after about 20 h of holding the pharmaceutical composition.

Without wishing to be bound by theory, it is assumed that gluconolactone—further to its action as a stabilizer—also acts as pH modifier: Through a ring opening reaction of gluconolactone, a carboxylic acid is slowly formed upon holding the pharmaceutical composition, which results in a decrease in pH.

### Example 3: Sterilization Method

For investigating the influence of the sterilization method on the stability of rocuronium bromide, a pharmaceutical composition comprising rocuronium bromide was prepared according to example 1, method 1 and left for 20 h or more for pH stabilization.

Then, the pharmaceutical composition was sterilized under different conditions, i.e.
a) filtering the pharmaceutical composition through two filters (Sartopore® 2 obtained from Sartorius Stedim, maximum pore size of 0.2 μm; referred to as “filtration”); or
b) terminal sterilization at 121° C. for a period F₀ of 8 minutes (referred to a “condition 1”); or
c) terminal sterilization at 121° C. for a period F₀ of 15 minutes (referred to as “condition 2”); or
d) terminal sterilization at 121° C. for a period F₀ of 20 minutes (referred to as “condition 3”).

The pharmaceutical compositions were tested for the amount of rocuronium bromide according to Assay (I), amount of impurities according to Assay (II), and pH before and after sterilization to evaluate the chemical stability of rocuronium bromide. Results are shown in Table 4.

TABLE 4
Results of stability studies of rocuronium bromide during sterilization.

| | non-filtered | | | | |
|---|---|---|---|---|---|
| Sterilization condition | control | filtration | condition 1 | condition 2 | condition 3 |
| pH | 3.9 | 3.9 | 3.9 | 3.8 | 3.8 |
| Rocuronium bromide [%] | 103.1 | 101.7 | 102.5 | 103.0 | 102.7 |
| Impurity C [%] | 0.1 | 0.1 | 0.3 | 0.5 | 0.5 |
| total unknown impurities [%] | n.d. | 0.1 | 0.1 | 0.1 | 0.1 |

n.d. = not determined

As can be concluded from the results shown in Table 4, filtration does not result in the formation of impurities, such as impurity C. Furthermore, the exposition to the material inside the Sartopore® 2 filters, i.e. polyethersulfone (PES) did not considerably change the amount or rocuronium bromide in the composition. In other words, no absorption was observed.

On the other hand, terminal sterilization (conditions 1, 2 and 3) resulted in the formation of impurity C which reduces shelf life of the compositions. Besides, also unknown impurities are formed.

Therefore, sterilization through filtration is considered advantageous.

### Example 4: Filling of Glass Syringes and Storage Stability Studies

For investigating the influence of the storage stability of rocuronium bromide, a pharmaceutical composition comprising rocuronium bromide was prepared according to example 3, wherein the pharmaceutical composition was sterilized by filtration as described in example 3.

The pharmaceutical composition was filled into Hylok Luer-Lock 5 mL glass syringes under a nitrogen atmosphere containing 4% (V/V) of oxygen, and stoppered with a rubber stopper. The glass syringes thus prepared contained 5.12 mL of the pharmaceutical composition to ensure a deliverable volume of 5 mL determined according to USP General chapter <657>, and a head space containing the nitrogen atmosphere containing 4% (V/V) of oxygen. The head space distance between the top of the liquid pharmaceutical composition and the lower edged of the plunger was 4.99 mm.

The glass syringes thus obtained were stored under different conditions (that are 25±2° C./60%±5% rH, 30±2° C./65%±5% rH, or 40±2° C./75%±5% rH, respectively). At different time points, the amount of rocuronium bromide and impurities were determined according to Assay (I) or Assay (II), respectively. Results are shown in Table 5, Table 6, and Table 7.

TABLE 5
Results of stability tests at 25 ± 2° C. / 60% ± 5% rH

| | storage time [months] | | | | |
|---|---|---|---|---|---|
| | 0 | 1 | 2 | 3 | 6 |
| pH | 3.8 | 3.9 | 3.9 | 3.8 | 3.8 |
| oxygen conc. | 3.4 | 5.0 | 5.8 | 6.8 | 5.3 |
| in headspace | | | | | |
| [% V/V] | | | | | |
| Rocuronium | 103.2 | 101.9 | 101.1 | 101.8 | 102.1 |
| bromide [%] | | | | | |
| Impurity C [%] | 0.2 | 0.3 | 0.4 | 0.6 | 1.3 |

TABLE 6
Results of stability tests at 30 ± 2° C. / 65% ± 5% rH

storage time [months]

| | 0 | 1 | 2 | 3 |
|---|---|---|---|---|
| pH | 3.3 | 3.8 | 3.8 | 3.8 |
| oxygen conc. | 5.4 | 6.9 | 8.1 | 7.1 |
| in headspace | | | | |
| [% V/V] | | | | |
| Rocuronium | 102.2 | 102.5 | 101.6 | 102.1 |
| bromide [%] | | | | |
| Impurity C [%] | 0.08 | 0.5 | 0.9 | 1.3 |

TABLE 7
Results of stability tests at 40 ± 2° C. / 75% ± 5% rH

| | storage time [months] | | | | |
|---|---|---|---|---|---|
| | 0 | 1 | 2 | 3 | 6 |
| pH | 3.8 | 3.9 | 3.9 | 3.9 | 3.8 |
| oxygen conc. | 3.4 | 5.8 | 7.1 | 8.4 | 9.3 |
| in headspace | | | | | |
| [% V/V] | | | | | |
| Rocuronium | 103.2 | 102.1 | 102.0 | 99.7 | 96.8 |
| bromide [%] | | | | | |
| Impurity C [%] | 0.2 | 0.7 | 1.4 | 2.4 | 5.6 |

As can be concluded from the results shown in Table 5 to Table 7, the amount of rocuronium bromide as well as pH remain constant over a period of 6 months, even when stored at elevated temperature and despite the presence of oxygen.

Therefore, it is concluded that a pharmaceutical composition prepared by the method of the present disclosure is highly stable and can thus be stored at room temperature, i.e. without the need for refrigeration.

1. A method for manufacturing a prefilled glass syringe comprising a sterile pharmaceutical composition of rocuronium bromide, the method comprising the steps of:
S1: providing a first amount of water for injection;
S2: dissolving at least one stabilizer in the water for injection provided in step S1 to provide a stabilizer solution;
S3: dissolving a buffer system in the stabilizer solution obtained from step S2 to provide a buffered stabilizer solution;
S4: dissolving rocuronium bromide in the buffered stabilizer solution obtained from step S3 and adding water for injection in an amount to provide a buffered rocuronium composition, wherein the buffered rocuronium composition comprises rocuronium in an amount of about 10 mg/ml;
S5: sterilizing the buffered rocuronium composition obtained from step S4 to provide a sterile pharmaceutical composition of rocuronium bromide;
S6: filling the sterile pharmaceutical composition of rocuronium bromide obtained from step S5 into a glass syringe under aseptic conditions; and
S7: blanketing the glass syringe obtained from step S6 with an inert gas and inserting a plunger into said glass syringe obtained from step S6 to provide the prefilled glass syringe comprising the sterile pharmaceutical composition of rocuronium bromide.

2. The method according to claim 1, wherein the glass syringe has a glass syringe barrel, a rubber stopper, a plunger rod, and a Luer Lock.

3. The method according to claim 1, wherein step S7 is carried out under an atmosphere of nitrogen and up to about 4% (V/V) of oxygen.

4. The method according to claim 1, wherein the at least one stabilizer is gluconolactone.

5. The method according to claim 1, wherein the buffer system comprises a pharmaceutically acceptable salt of citric acid, or a pharmaceutically acceptable salt of acetic acid, or a pharmaceutically acceptable salt of glycine, or a mixture thereof.

6. The method according to claim 1, wherein the at least one stabilizer and the buffer system are added in such amount that the buffered rocuronium composition obtained from step S4 has a pH of about 3.8 to about 4.2.

7. The method according to claim 6, wherein the at least one stabilizer is gluconolactone and the buffered rocuronium composition obtained from step S4 comprises gluconolactone in an amount of about 25 mg/mL.

8. The method according to claim 6, wherein the buffer system is a mixture of sodium citrate·2 H₂O and sodium acetate·3 H₂O.

9. The method according to claim 8, wherein the buffered rocuronium composition obtained from step S4 comprises sodium citrate·2 H₂O in a concentration of about 5 mg/mL and sodium acetate·3 H₂O in a concentration of about 5 mg/mL.

10. The method according to claim 6, wherein step S4 comprises mixing the buffered rocuronium composition for at least 20 h until a stable pH is obtained.

11. The method according to claim 1, wherein the buffered rocuronium composition obtained from step S4 has an osmolarity of about 270 mOsmol/kg to about 310 mOsmol/kg.

12. The method according to claim 11, wherein any one of steps S2, S3, or S4 further comprises adding a tonicity agent, such as sodium chloride.

13. The method according to claim 1, wherein S5 comprises filtering the buffered rocuronium composition obtained from step S4 through a double layer filter, wherein a first layer of the double layer filter has a pore size of about 0.2 μm or about 0.45 μm, and a second layer of the double layer filter has a pore size of about 0.2 μm.

14. The method according to claim 1, wherein step S1 further comprises sparging the water for injection with nitrogen for at least 15 min.

15. The method according to claim 14, wherein sparging is performed at a pressure of about 5 psi (about 345 mbar).

16. The method according to claim 1, wherein steps S2, S3, S4, and S5 further comprise sparging with nitrogen after each addition.

17. The method according to claim 1, wherein the sterile pharmaceutical composition of rocuronium bromide comprised in the prefilled glass syringe obtained from step S7 comprises at most 0.2% of total impurities, as determined by HPLC.

18. The method according to claim 1, wherein the sterile pharmaceutical composition of rocuronium bromide comprised in the prefilled glass syringe contains at least 9.8 mg/mL of rocuronium bromide after storage at 25° C. for up to 6 months, as determined by HPLC.

19. The method according to claim 1, wherein the sterile pharmaceutical composition of rocuronium bromide comprised in the prefilled glass syringe contains at most about 1.3% of total impurities after storage at 25° C. for up to 6 months, as determined by HPLC.

20. The method according to claim 1, wherein in step S6 a volume of the sterile pharmaceutical composition of rocuronium bromide obtained from step S5 is adjusted in that a deliverable volume of the sterile pharmaceutical composition of rocuronium bromide is about 5 mL to about 10 mL, wherein the deliverable volume is determined according to USP General chapter <657>.

21. The method according to claim 1, wherein the prefilled glass syringe comprising the sterile pharmaceutical composition of rocuronium bromide further comprises a gas phase, wherein the gas phase has an oxygen content of up to about 4% (V/V).

22. A prefilled glass syringe comprising a sterile pharmaceutical composition of rocuronium bromide obtained by the method according to claim 1.

23. A kit comprising:
a prefilled glass syringe comprising a sterile pharmaceutical composition of rocuronium bromide obtained by the method according to claim 1;
a plunger rod; and
a carton box as secondary packaging.
